(19) United States
(12) Patent Application Publication
(10) Pub. No.: US 2014/0302113 A1
(43) Pub. Date: Oct. 9, 2014

(54) SYSTEMS, DEVICES, AND METHODS FOR EMBEDDING DRUG MOLECULES INTO MEDICAL CATHETERS OR TUBES

(71) Applicant: SparkMed Research, LLC, Salt Lake City, UT (US)

(72) Inventor: Jie Zhang, Salt Lake City, UT (US)

(21) Appl. No.: 14/347,749

(22) PCT Filed: Sep. 28, 2012
(86) PCT No.: PCT/US12/58137

§ 371 Date: Mar. 27, 2014

Related U.S. Application Data
(60) Provisional application No. 61/541,594, filed on Sep. 30, 2011.

(51) Int. Cl. A61L 29/16; A61L 29/08

(52) U.S. Cl. CPC A61L 29/16; A61L 29/08; A61L 2420/02; A61L 2300/402; A61L 2300/602; A61L 2300/606; A61L 2300/204; A61L 2300/206; A61L 2300/404; A61L 2300/45
USPC 424/423; 514/626; 514/635; 427/2.3

(57) ABSTRACT

The present disclosure relates to methods for embedded drug molecules into medical catheters, tubes, and other medical devices. The catheter, tube, or other medical device is capable of releasing drugs for extended periods of time. Drugs can be loaded into the wall thereof through diffusion from a loading solution. A counterintuitive approach of using undissolved drug particulates in the loading solution is employed in some embodiments. The drug in the wall of the device and in the loading solution can be in dynamic equilibrium, yielding stable and easy-to-manufacture products. Heat can be used to significantly speed up the drug loading.

### BACKGROUND

Medical catheters, drainage tubes such as urinary (Foley) catheters, and other tubes such as tracheal tubes and central venous catheters, are often used on sensitive mucosal or wound surfaces and can cause pain, discomfort, and/or infection. For example, a Foley catheter, which is a soft, thin rubber tube with a balloon at one end to hold it in place within the bladder, is often used to drain urine from the bladder. It can remain in place for a short or long period of time, and is inserted through the urinary duct (urethra) and into the bladder. As a Foley catheter is used typically when normal urination is disrupted by an infection, a swollen prostate gland, bladder stones, injury, post-surgical period, or the like, the use of this device in this already sensitive area can be uncomfortable or painful or become a foreign surface on which biofilm or other microbes can grow. Biofilm in particular, in which bacteria can hide, can form on the surfaces of the catheters or tubes that are in contact with mammalian mucosa, wound, or other tissues for longer than a few days. Thus, long term use of Foley catheter is often associated with infection in the urinary tract. Furthermore, the use of central venous catheters sometimes causes dangerous infections. As a result of this and other shortcomings, there is a need to provide medical devices and methods of using them with a decreased degree of discomfort and/or decreased possibility of infections.

### BRIEF DESCRIPTION OF THE DRAWINGS

FIG. 1 is a schematic drawing of a urinary (Foley) catheter which can be used in accordance with embodiments of the present disclosure; and

FIG. 2 is a schematic drawing of an embodiment of the present disclosure which includes a catheter, a closed container, a loading solution, and a sealing mechanism; and

FIG. 3 is a schematic drawing of another embodiment of the present disclosure which includes a catheter, a container, a loading solution, and a fluid retention ring assists in closing the container when the catheter is in place.

### DETAILED DESCRIPTION

Reference will now be made to the exemplary embodiments and specific language will be used herein to describe the same. It will nevertheless be understood that no limitation of the scope of the disclosure is thereby intended. Alterations and further modifications of the inventive features illustrated herein, and additional applications of the principles of the disclosure as illustrated herein, which would occur to one skilled in the relevant art and having possession of this disclosure, are to be considered within the scope of the disclosure. It is also to be understood that this disclosure is not limited to the particular configurations, process steps and materials disclosed herein as these may vary to some degree. Further, it is to be understood that the terminology used herein is used for the purpose of describing particular embodiments only, and is not intended to be limiting as the scope of the present disclosure.

It is noted that, as used in this specification and the appended claims, singular forms of “a,” “an,” and “the” include plural referents unless the content clearly dictates otherwise.

“Medical catheter,” “medical tube,” “catheter,” and “tube” are used interchangeably and are defined as any catheter or tube used for medical purposes, typically in humans or other mammals. These catheters and tubes include, without limitation, urinary tract catheters (Foley catheter), drainage tubes, feeding tubes, trachea tubes, intravenous catheters, central venous catheters, arterial catheters, umbilical (arterial and venous) catheters, gastric tubes, uterine tubes, chest tubes, peritoneal catheters, renal catheters, dialysis catheters, tissue drainage tubes, or any medical catheter or tube that, when in use, has a surface in contact with mammalian mucosa, wound, or other tissue not protected by skin. This term also includes other medical devices that are used in invasive medical procedures, such as laparoscopic and endoscopic instruments. In general, the medical catheters or tubes that are within the scope of the present disclosure include any medical device that has a surface in contact with a human tissue not protected by normal skin with intact stratum corning layer. These tissues can include mucosa, wound tissue, and internal organ tissue.

“Foley catheter” and “urinary catheter” are used interchangeably.

“Rubber” when used to describe the wall material of a medical catheter or tube means any elastic or soft material typically used to make the wall or part of the wall of a medical catheter or tube, and includes, without limitation, silicone, latex, polyurethane, copolymers having urethane monomer units, and the combination thereof. “Rubber” can be used interchangeably with “polymer” in the present disclosure.

“Drug” or “drugs” are substances that can be used to treat or prevent diseases, pain, infection, inflammation, or discomfort, such as in humans or other mammals. Examples include substances that can reduce or eliminate pain or reduce the possibility or severity of infection, e.g., local anesthetic agents, anti-infection agents including anti-bacterial, anti-viral, anti-fungal, anti-biofilm formation agents, etc., opioids, anti-inflammatory agents, and the like.

“Local anesthetic agent(s)” includes both amide type local anesthetic agents such as lidocaine, prilocaine, articaine, bupivacaine, dibucaine, etidocaine, levobupivacaine, mepivacaine, piperocaine, ropivacaine, trimecaine, as well as ester type local anesthetic agents such as such as benzocaine, chloroprocaine, cocaine, cyclomethycaine, dimethocaine, propoxycaine, procaine, proparacaine, and tetracaine. In one embodiment, the local anesthetic agent used can be in its base form.

“Anti-infection agent(s)” includes all substances that are capable of preventing viral, fungal, or bacteria infections or reducing the severity of such infections, such as, without limitation, antibiotics, metallic anti-infectives such as a silver compound, and the like. “Anti-infection agent(s)” also includes substances that prevent or inhibit the formation of biofilm on the surfaces of medical catheters, tubes, and devices. Thus, anti-infection agents include, without limitation, chlorhexidine (which by definition herein includes its salts such as chlorhexidine diacatea or chlorhexidine gluconate), silver sulfadiazine, triclosan, nitrofurazone, minocycline, rifampicin, ciprofloxacin, fosfomycin, vancomycin, tobramycin, cefamandol, cephalothin, carbenicillin, amoxicillin, gentamicin, flucloxaxillin, ceragenins, flucanazole, furanone, echinocandins, amphotericins, gendine, chitosan, IgG, cephalosporin, ethylenediamine tetraacetic acid (EDTA), metal binding chelators, and/or biofilm inhibiting peptides.

“Silver compound” is a substance that contains the element silver, and can include substances that produce silver ions in a solution comprising water. Silver compounds include, without limitation, silver nitrate, silver sulfadiazine, silver oxide, and colloidal silver including elemental colloids of silver and alloys thereof. Anti-inflammatory agents include, without limitation, non-steroidal anti-inflammatory drugs (NSAIDS) such as ketoprofen, diclofenac, Ibuprofen, Indomethacin, salicylates, mefenamic acid, meloxicam, nabumetone, naproxen, oxaprozin, piroxicam, rofecoxib, sulindac, valdecoxib, and their salts. Anti-inflammatory agents also include certain steroids such as Hydrocortisone, hydrocortisone acetate, cortisone acetate, tixocortol pivalate, prednisolone, methylprednisolone, prednisone, Triamcinolone acetonide, triamcinolone alcohol, mometasone, amcinonide, budesonide, desonide, fluocinonide, fluocinolone acetonide, halcinonide, betamethasone sodium phosphate, dexamethasone, dexamethasone sodium phosphate, fluocortolone, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate, fluprednidene acetate, h, 17-aceponate, 17-buteprate, and prednicarbate.

Opioids include, without limitation, cocaine, morphine, fentanyl, miperidine, oxycodone, hydrocodone, codeine, hydromorphone, buprenorphine, methadone, sufentanil, remifentanil, and tramadol. While some opioids, such as cocaine, can produce local anesthetic effect, the main acting site of opioids is in the brain. Therefore, controlled extended release of an opioid drug from a catheter or tube surface on a patient's wound or mucosal surface releases the drug into the patient's systemic circulation, and the drug eventually enters the brain, in a controlled and extended fashion.

“Loading solution” is a solution used to load the drug into the wall of a catheter or tube. In a typical use, the drug can be placed (but not necessarily completely dissolved) in a loading solution. The catheter or the tube (or at least the part of its surface intended for drug release) can be immersed into the loading solution for a period of time so that the drug in the loading solution can be absorbed by the wall of the catheter or tube. In one embodiment, as the drug becomes absorbed by the wall of the catheter or tube, additional undissolved drug in the loading solution can become dissolved in the loading solution, thereby providing additional dissolved drug for further absorption in the wall of the catheter or tube.

“Therapeutically effective,” is defined as an effect on a mammalian (typically human) subject that is statistically significant (p<0.05) when tested in 12 or more subjects.

The phrases “relief of pain,” and “clinically relevant reduction of pain” or reduction in “discomfort” or “sensitivity” have the same meaning and can be used interchangeably, and can be defined as an average reduction of 3 points or more from the baseline on an 11-point (0-10) numeric pain rating scale compared with placebo when tested using at least 12 subjects.

The unit “mcg/hour/cm²” means microgram per hour per square centimeter.

“Extended period of time” is defined as a period of at least 24 hours.

The phrase “capable of releasing a drug for an extended period of time” (or similar phrases) means capable of releasing the drug at therapeutically effective rates for an extended period of time. For a local anesthetic agent, this is defined as being capable of releasing the local anesthetic agent at rates sufficient to numb targeted mammalian tissue for that period of time. For lidocaine, this means rates higher than 1 mcg/hour/cm², typically at least 3 mcg/hour/cm², at least 5 mcg/hour/cm², and often at least 10 mcg/hour/cm². For tetracaine, this can be rates of at least 1 mcg/hour/cm², for example. Other local anesthetics can be used providing similar tissue numbing as would be apparent to one skilled in the art after considering the present disclosure. For anti-infection or anti-inflammatory substances, this phrase means releasing of an anti-viral, antibacterial, anti-fungal, anti-biofilm, anti-inflammatory agent, or opioid at an effective rate for at least 2 days, at least 3 days, and often at least 5 days or at least 7 days.

The word “solution” includes liquid (solvent or solvents) containing partially or completely dissolved solid (solute).

The terms “absorb,” “load” or “embed” include loading a drug into the wall material of a catheter or tube by letting the drug diffuse into the wall material (as opposed to merely coating a layer of material containing the drug onto the surface of the catheter or tube).

“Wall” describes the body of the catheter or tube that can be exposed to loading solution when the catheter is submerged in the loading solution. “Outer wall surface” refers to the surface that typically contacts the body when the tube or catheter is inserted and “inner wall surface” refers to the functional surface where fluid is typically passed in or out.

“Wall concentration” or “surface concentration”, used interchangeably, means quantity of a substance (e.g. a drug) absorbed into each unit surface area of the wall of the catheter or tube (The wall surface area is measured from the surface that is in contact with the loading solution). For example, if 5 mg of lidocaine is absorbed through a one square centimeter area of the outer wall surface of a Foley catheter, that area of the wall has a wall concentration of lidocaine of 5 mg/cm², regardless how deep the lidocaine is distributed within the wall material. For example, if a Foley catheter has a outer diameter of 0.6 cm and 236 mg of lidocaine has been absorbed into the wall through a 25 cm length section of the catheter's outer wall surface, the average wall concentration of lidocaine in the 25 cm length section of the wall is 236 mg/(0.6 cm×π×25 cm)=5 mg/cm². The wall concentration of a drug, as used in the present disclosure, includes only the drug actually absorbed into the wall material and does not include the drug merely sits on the surface of the wall (e.g., that can be removed by a simple wipe).

The terms “immerse” or “submerge” when used to describe keeping the wall of a catheter or tube in the loading solution, includes not only the immersion of the catheter or tube in the loading solution, but also, maintaining part, most, or all of the outer wall of the catheter or tube in the loading solution for a sufficient period of time to allow for drug loading into the walls of the catheter or tube.

The term, “dynamic equilibrium” when used to describe the distribution of a drug in the solution and in the catheter wall includes the situation in which both the drug distribution within the catheter wall material and the distribution between the loading solution and the wall material have reached equilibrium (so that there is no net inflow or outflow of the drug into the wall). It can also include the situation in which the dynamic equilibrium between the solution and wall surface is almost reached except there is still a slight inflow of the drug into the wall due to the diffusing of the drug already in the wall material deeper into the wall material. Thus “dynamic equilibrium” can refer to a situation where the drug in solution and the wall are in “complete” equilibrium, or where there is “substantially complete” equilibrium with only minimal or slight diffusion into the wall. In “substantially complete” equilibrium, the slight drug inflow is defined as a positive drug inflow into the wall of the catheter or tube at a rate of less than 10 wt % of the peak inflow rate after the catheter is first submerged in the loading solution. In both cases, the tube with drug loaded in the wall thereof will be effective for use in accordance with embodiments of the present disclosure

When referring to “mucosa,” “mucosal tissue,” or “mucosal surface,” this includes mammalian mucosal, sub-mucosal, peritoneal, or any other kind of wound or similar type of tissue.

The term “patient” or “subject” refers to humans and other mammals.

As used herein, a plurality of drugs, and/or substances may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range.

It is noted that all percentages are in weight, unless specified otherwise.

The present disclosure is related to methods for embedding drug molecules into the wall material of medical catheters and tubes, so that when the catheter or tube is used in the patient, the drug molecules can be released over an extended period of time to reduce the discomfort and/or possibility of infections. The present disclosure is also related to methods for storing the drug-embedded medical catheters and tubes.

With this background in mind, a medical catheter or tube capable of releasing drugs for extended period of time for reducing pain and/or infection, as well as methods of making the same are provided. In one embodiment, a method of loading sufficient amount of a local anesthetic agent into a medical catheter or tube for releasing the local anesthetic agent for at least 7 days when used in a patient is disclosed. The medical catheter or tube can comprise an outer wall surface and an inner wall surface, at least a portion of the outer wall surface having a wall area with a wall concentration of a local anesthetic agent of at least 1 mg/cm². The local anesthetic agent can be diffused into material of the wall via a loading solution. The diffusion process can be significantly accelerated with increased temperatures.

In another embodiment, a system for loading a drug into the wall of a medical catheter or tube capable of releasing a local anesthetic agent for at least 24 hours or at least seven days when used in a patient can comprise a medical catheter or tube comprising wall material having an outer wall surface and an inner wall surface, and a loading solution, including a local anesthetic agent, in which at least a portion of the outer wall surface has been submersed in the loading solution under heat. More specifically, a first portion of the local anesthetic agent can be in the loading solution and a second portion the local anesthetic agent can be absorbed into the wall material of the medical catheter or tube. In one specific embodiment, at least a part of the wall material can have, absorbed therein, a sufficient amount of the local anesthetic agent from the loading solution to have a wall concentration of a local anesthetic agent of at least 1 mg/cm², or at least 4 mg/cm². In another specific embodiment, the local anesthetic agent in the catheter or tube wall and in the loading solution is in dynamic equilibrium. In another specific embodiment, the local anesthetic agent in the wall material of the medical catheter or tube and in the loading solution are in dynamic equilibrium.

In another embodiment, a system for loading a drug into the wall of a medical catheter or tube capable of releasing a local anesthetic agent for at least 24 hours, or at least seven days, when used in a patient can comprise a medical catheter or tube comprising wall material having an outer wall surface and an inner wall surface, and a loading solution, including a local anesthetic agent, in which at least a portion of the outer wall surface has been submersed in the loading solution and the temperature of the loading solution is at least 35° C., at least 40° C., or at least 50° C., at least 80° C., at least 100° C., and/or at a temperature high enough for autoclave sterilization. In one specific example, the outer wall surface can be submerged in said loading solution about 20 minutes or more when the temperature of the loading solution is about 100° C. or 105° C., or at least 24 hours when the temperature of the loading solution is at least about 50° C. More specifically, a first portion of the local anesthetic agent can be in the loading solution and a second portion the local anesthetic agent can be absorbed into the wall material of the medical catheter or tube. In one specific embodiment, at least a part of the wall material can have, absorbed therein, a sufficient amount of the local anesthetic agent from the loading solution to have a wall concentration of a local anesthetic agent of at least 1 mg/cm². In another specific embodiment, the local anesthetic agent in the catheter or tube wall and in the loading solution is in dynamic equilibrium. In another specific embodiment, the local anesthetic agent in the wall material of the medical catheter or tube and in the loading solution are in dynamic equilibrium.

In another embodiment, a medical catheter or tube capable of releasing a local anesthetic agent for at least 24 hours, or at least 7 days, when used in a patient can comprise a wall area having a wall concentration of a local anesthetic agent of at least 1 mg/cm². The local anesthetic can be selected from the group consisting of lidocaine, prilocaine, articaine, bupivacaine, dibucaine, etidocaine, levobupivacaine, mepivacaine, piperocaine, ropivacaine, trimecaine, benzocaine, chloroprocaine, cocaine, cyclomethycaine, dimethocaine, propoxycaine, procaine, proparacaine, tetracaine, and mixtures thereof.

In another embodiment, a system of a loading solution and a medical catheter or tube capable of releasing a local anesthetic agent for at least 24 hours, or at least 7 days, when used in a patient can comprise a medical catheter or tube comprising wall material having an outer wall surface and an inner wall surface, and a loading solution in which at least a portion of the outer wall surface has been submersed and heated to temperatures as described herein. The loading solution can include a local anesthetic agent selected from the group of lidocaine, prilocaine, articaine, bupivacaine, dibucaine, etidocaine, levobupivacaine, mepivacaine, piperocaine, ropivacaine, trimecaine, benzocaine, chloroprocaine, cocaine, cyclomethycaine, dimethocaine, propoxycaine, procaine, proparacaine, tetracaine, and mixtures thereof. In this embodiment, a first portion of the local anesthetic agent remains in the solution and a second portion of the local anesthetic agent is absorbed into the wall material. At least a part of the wall material can have a wall concentration of the local anesthetic agent of at least 1 mg/cm².

In still another embodiment, a method of making a medical catheter or tube capable of releasing a drug for at least 24 hours can comprise placing at least a portion of a medical catheter or tube into a loading solution so that an outer wall surface contacts the loading solution, the loading solution having an initial pH such that more than 50% of the drug in it exists in an unionized form, e.g., the molecule carries no net charge, (some part of the local anesthetic agent may not be dissolved in the solution. This approach may be considered counter intuitive because a drug typically has low solubility in a solution with such a pH, and most of the drug may exist as undissolved particles, which cannot diffuse into the wall directly (only the molecules dissolved in the solution can diffusion into the wall).

In another embodiment, a method of making a medical catheter or tube capable of releasing a local anesthetic agent for at least 24 hours or at least seven days can comprise placing at least a portion of a medical catheter or tube into a loading solution so that an outer wall surface contacts the loading solution, the loading solution having a pH higher than about 7.0 and comprising a local anesthetic agent and more than 50% water by weight; and heating as described herein with the outer wall surface in contact with the loading solution before using the medical catheter or tube for a medical purpose.

In another embodiment, a method for loading a drug into the a surface wall of a medical device can comprise keeping a surface of a medical device in a loading solution comprising a drug under heat, wherein more than 50% of the drug molecules exist in unionized form in the loading solution initially when the loading process begins. The drug can include, without limitation, lidocaine and/or chlorhexidine. The unionized drug in the solution may exist as undissolved particles, for example.

In another embodiment, a method for loading a drug into the a surface wall of a medical device can comprise keeping a surface of a medical device in a loading solution comprising a drug for at least 24 hours and maintaining the temperature of the solution at least at 50° C. for at least 24 hours, wherein more than 50% of the drug molecules exist in unionized form in the loading solution initially when the loading process begins.

In another embodiment, a medical device capable of releasing a drug for at least 24 hours when used in a patient can comprise an outer wall surface having a wall area with a wall concentration of a drug of at least 1 mg/cm². The drug is diffused into material of the wall via a loading solution.

In another embodiment, a medical device capable of releasing a chlorhexidine for at least 24 hours when used in a patient can comprise a wall having a wall area with a wall concentration of chlorhexidine of at least 20 mcg/cm²′, or at least 100 mcg/cm². The drug is diffused into material of the wall via a loading solution.

The weight of chlorhexidine in the present disclosure is expressed in the equivalence of chlorhexidine diacetate, unless expressed otherwise. For example “1 mg chlorhexidine” means the amount of chlorhexidine in 1 mg of chlorhexidine diacetate, regardless if the chlorhexidine is in salt, ion, or free base form.

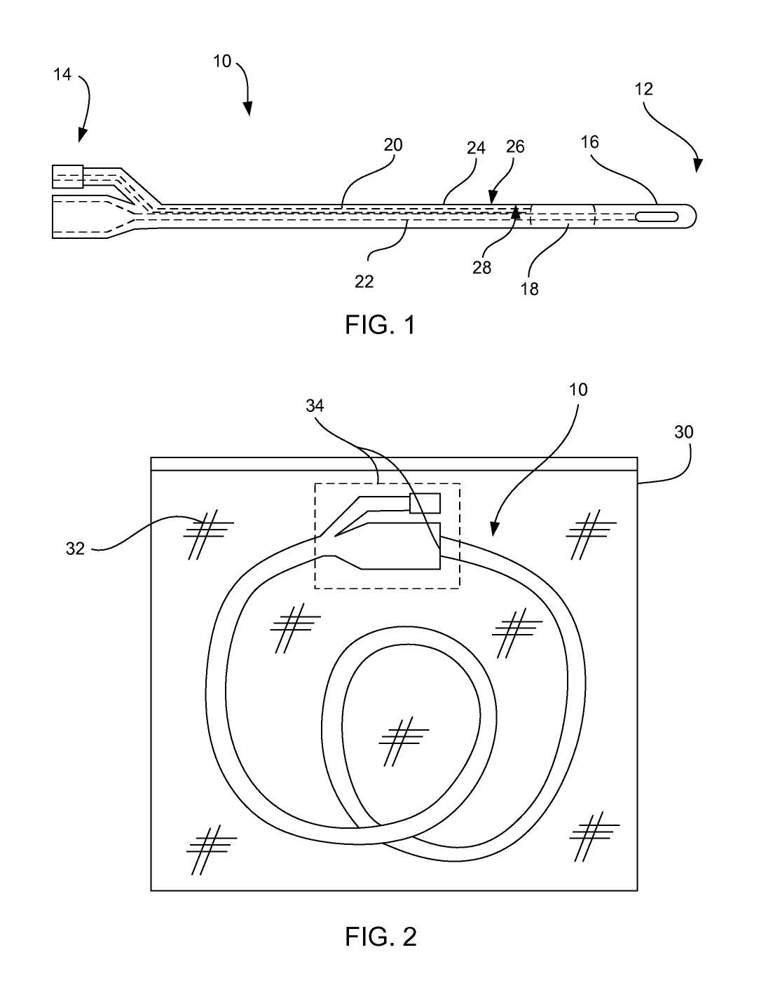

In each of these embodiments, reference is made to a medical catheter or tube, or a medical device. For exemplary purposes, a urinary (Foley) catheter is shown by way of illustration in FIG. 1. However, other medical catheters and tubes can likewise be similarly prepared, and it is noted that the Foley catheter is shown merely to illustrate one possibility in accordance with examples of the present disclosure.

Specifically in FIG. 1, a urinary catheter 10 is shown which includes a patient end 12, and a discharge end 14. Close to the patient end is a patient end opening 16 and a balloon 18. The balloon is inflated with air or filled with a liquid through a channel 20 once the patient end is inserted into the bladder of the patient, which keeps the device from being inadvertently removed. When in place, urine can drain from the bladder through the patient end opening 16, the urinary channel 22, and the discharge end opening. The urinary catheter has a wall 24, an outer wall surface 26, and inner wall surface 28. Other modifications of this type of catheter can also be used in accordance with embodiments of the present disclosure. For example, there are catheter systems with three channels instead of two, there are variously configured balloons, and variously configured openings and channels, etc. The mechanical configuration of the specific catheter is not necessarily part of the invention in every embodiment, as the drugs of the present disclosure can be used in many differently configured medical catheters and tubes, and even devices. Thus, it is noted that many different types of tubes can be used in accordance with embodiments of the present disclosure.

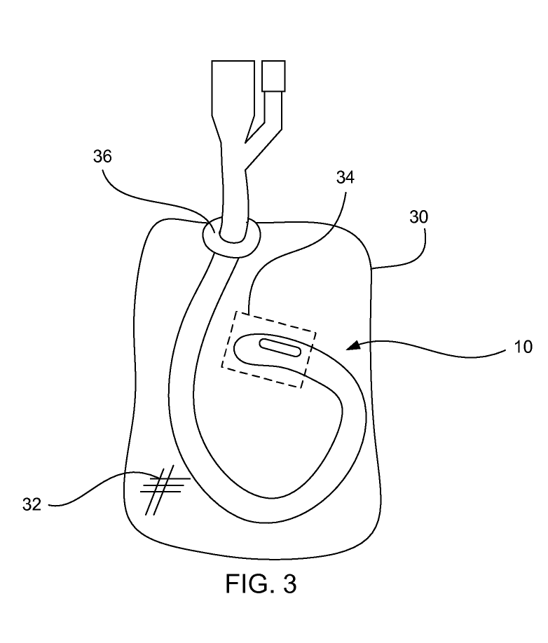

In each of the following embodiments, which are related to the embodiments described above and elsewhere herein, reference is made to a medical catheter or tube, or a medical device. For exemplary purposes, a urinary (Foley) catheter is shown by way of illustration in FIG. 1, and a system in accordance with embodiments of the present disclosure is shown in FIGS. 2 and 3. However, other medical catheters and tubes can likewise be similarly prepared, and it is noted that the Foley catheter is shown merely to illustrate one possibility in accordance with examples of the present disclosure.

Specifically in FIG. 1, a urinary catheter 10 is shown which includes a patient end 12, and a discharge end 14. Close to the patient end is a patient end opening 16 and a balloon 18. The balloon is inflated with air or filled with a liquid through a channel 20 once the patient end is inserted into the bladder of the patient, which keeps the device from being inadvertently removed. When in place, urine can drain from the bladder through the patient end opening 16, the urinary channel 22, and the discharge end opening. The urinary catheter has a wall 24, an outer wall surface 26, and inner wall surface 28. Other modifications of this type of catheter can also be used in accordance with embodiments of the present disclosure. For example, there are catheter systems with three channels instead of two, there are variously configured balloons, and variously configured openings and channels, etc. The mechanical configuration of the specific catheter is not necessarily part of the invention in every embodiment, as the drugs of the present disclosure can be used in many differently configured medical catheters and tubes, and even devices. Thus, it is noted that many different types of tubes can be used in accordance with embodiments of the present disclosure.

FIG. 2 shows the urinary catheter 10 in a rolled up configuration, and contained within a sealed container 30. Within the sealed container is a loading solution 32 which is described herein at length. Also shown is a schematic of a sealing mechanism 34, particularly its location and purpose is for protecting the urinary channel from being filled with the loading solution (it is noted that in some embodiments the urinary channel can be filled with the loading solution for faster and/or more even loading of the drug into the wall. In those embodiments, the loading solution can be drained from the urinary channel before the catheter is used in the patient). In this particular embodiment, the patient end 12 is inserted into the urinary channel 22 at the discharge end 14, covering the patient end opening (not shown). The sealing mechanism can be used to seal the insertion point between the two ends such that when the catheter is submerged into a solution, little to no solution can get to inside of the catheter. Other systems can also be utilized, with the primary goal of preventing loading solution from substantially entering the urinary channel, thereby becoming clogged prior to use.

Referring now to FIG. 3, a urinary catheter 10 is shown in a rolled up configuration and partially contained within a sealed container 30. Within the sealed container is a loading solution 32. Also shown is a schematic of a sealing mechanism 34 which seals an opening at the end of the catheter. In addition to the sealing mechanism, this embodiment also includes a fluid retention ring 36. Specifically, the loading solution can be loaded within the container, and the catheter can be positioned through fluid retention ring and partially within the container. In this configuration, the fluid is retained within the sealed container, as the catheter provides a temporary seal in combination with the fluid retention ring. To remove the catheter for use, the user can simply pull the exposed end of the catheter from the sealed container (through the fluid retention ring) and the catheter is ready to use with little to no further preparation.

Turning to additional embodiments, a system of the present disclosure is prepared in accordance with the following process: a Foley catheter (whose entire or part of the wall material is rubber, e.g., either silicone or latex or a combination thereof, is immersed into a water-based solution containing mostly unionized lidocaine (a major part of this lidocaine may initially exist as undissolved particles). The pH of the solution is higher than 6.5, and typically higher than about 7.0, and most typically higher than about 7.5. For a lidocaine in water solution, for example, solubility and percentage of ionized molecules decreases as the pH increases. As the wall of the catheter absorbs the unionized lidocaine dissolved in the solution, lidocaine in the undissolved (solid) form continues to dissolve into the solution. Thus, as dissolved drug is absorbed into the catheter wall, additional undissolved drug can get dissolved into the solution to keep the concentration of dissolved drug at or near the maximum allowed by the solubility, thereby providing more drug in the solution to cause it to continue being driven into the catheter wall. The temperature of the solution can be increased (e.g., greater than or equal to 40° C., greater than or equal to 50° C., greater than or equal to 60° C., or even greater than or equal to 100° C.) for a period of time (e.g. 15 minutes to 100 hours, depending on the temperature) to increase the speed of lidocaine absorption into the catheter wall. This approach is highly counterintuitive because only dissolved drug can be absorbed by the wall material, and a typical person skilled in the art would not deliberately formulate a solution in which most of the drug is undissolved. Additionally, it is noted that the above approached can be used for loading drugs other than lidocaine under the same rationale.

After the desired amount of lidocaine is absorbed into the catheter wall, the catheter is retrieved from the solution, dried, and stored. When it comes the time to use it, it is inserted into the urinary tract of the patient in the same way as an ordinary Foley catheter (usually with the help of an ordinary lubricant which has no drug-releasing function). Once the catheter is in the urinary tract, lidocaine in the rubber wall slowly diffuses out of the wall and into the mucosal surface in contact with the catheter wall. The mucosal surface is numbed by the continuous presence of lidocaine until the lidocaine in the catheter wall is depleted to the point that the lidocaine release rate drops to below the therapeutically effective rate.

In another more specific embodiment, a system of the present disclosure is prepared in accordance with the following process: a Foley catheter is immersed into a water-based solution (optionally viscous) containing lidocaine base solid (so the lidocaine base quantity in the solution is more than what the solution can dissolve). The pH of this solution with lidocaine is typically higher than about 7.0, or even higher than 7.5, such that most of the lidocaine in the solution exists in the unionized form and initially as undissolved particles in the solution. The solution, with the catheter in it, is then sealed into a closed container which is then placed into storage or at a location with increased temperature over standard room temperature. The rubber wall of the catheter starts to absorb the lidocaine dissolved in the solution. As the dissolved lidocaine gets absorbed into the catheter wall, lidocaine in the undissolved form in the solution continues to dissolve into the solution. After sufficient time (typically days or weeks at room temperature but can be as short as less than one hour at elevated temperatures), the wall of the Foley catheter has absorbed enough lidocaine for the purpose of extended release of lidocaine. However, the catheter continues to stay in the solution (which is in the sealed container) for the entire or most of the duration of storage prior to use. Sometime after the catheter is placed into the loading solution, the lidocaine in the solution and that in the wall of the catheter reach dynamic equilibrium (meaning the rate of lidocaine diffusing from the solution into the catheter wall equals the rate of lidocaine diffusing out of the catheter wall into the solution). As a result, there is no net lidocaine permeation into or out the catheter wall after the dynamic equilibrium is reached. Of course, this dynamic equilibrium between of the solution and catheter wall can be altered as the lidocaine already absorbed into the catheter wall diffuses deeper into the wall material. However, eventually, the lidocaine distribution inside the wall material will also reach equilibrium. At that point, the distribution of lidocaine in the wall material and in the solution will not change anymore. The quantity of lidocaine solid initially in the solution can be such that when the dynamic equilibrium is reached, there is no more solid lidocaine in the solution. Optionally, the quantity of lidocaine solid initially in the solution can be such that when the dynamic equilibrium is reached, there is no more solid lidocaine in the solution and the concentration of dissolved lidocaine in the solution is at or very close to the saturation point (as measured at 25° C.). When the concentration of unionized lidocaine dissolved in the solution is close to the saturation point of the unionized lidocaine (i.e. higher than 90% of the saturation concentration), the dynamic equilibrium is reached, the amount of lidocaine loaded into the catheter wall is close to the maximum amount that can be loaded with this method, and thus the catheter can provide maximum or near maximum duration of effective drug release when the catheter is used in a patient.

Optionally, the loading solution can also have the appropriate viscosity and other properties so that it can be used as lubricant for facilitating the insertion of the catheter into the patient's urinary tract. For example, the viscosity of the solution can be in the range of 300 to 600,000 centipoise, and often in the range of 3,000 to 150,000 centipoise. When it comes the time to use such a system, the openings are unblocked (if they were blocked) and there may be no need to smear additional lubricant onto the catheter before it is inserted into the patient's urinary tract because the viscous solution that is already on the surface of the catheter wall when it is retrieved from the loading solution and can perform the function of the lubricant. Once the catheter is in the urinary tract, lidocaine in the wall slowly diffuses out of the wall and into the mucosal surface in contact or in close proximity with the catheter wall. The mucosal surface is numbed by the continuous presence of lidocaine until the lidocaine release rate from the wall is decreased to a point below a clinically effective release rate, e.g., when the device stops providing its pain reducing benefits. The catheter in this embodiment can reduce the sensitivity of a part of or entire urinary tract for at least 24 hours, at least 72 hours, or at least 7 days. This method of loading the drug into the catheter wall as described above can be referred to as a dynamic equilibrium loading method (or technology).

The time it takes to reach the dynamic equilibrium depends on several factors, including the initial drug concentration in the loading solution, the loading solution temperature, the catheter wall material, etc. It is typically days or weeks at room temperature or slightly higher temperatures, e.g., greater than or equal to 40° C. or higher, but can be as short as hours or even a fraction of one hour if the temperature is higher enough. High temperatures can be effective in some examples, provided the temperature does not damage the integrity of the wall of the medical catheter or tube.

One advantage of the dynamic equilibrium loading method of the present disclosure is that the process of reaching dynamic equilibrium can take place automatically without significant work to be carried out by the manufacturing personnel. To shorten the time to reach dynamic equilibrium, the system can be placed in a higher temperature environment for a certain period of time. In a more general embodiment, the higher temperature range can be from 35° C. to 125° C., from 40° C. to 115° C., and often from 50° C. to 70° C. In a particular embodiment, sufficient amount of the drug can be embedded within two hours or even less than one hour by increasing the temperature of the solution and the catheter to a point high enough to sterilize the catheter and the solution. With this approach, the loading of the drug and the sterilization of the product can be carried out as part of the same process. Dynamic equilibrium can be reached during the heating step, or alternatively, can be reached during storage after heating is completed.

Similar to the preparation of the Foley catheter above, in one specific embodiment, a drainage system can be prepared in accordance with the following process: a drainage tube is immersed into a water-based solution with such a pH that most of the lidocaine in it is in the unionized form. The tube and the solution are then sealed into a closed container. The container has an opening to accommodate a sealing ring whose size and shape matches that of the cross-section of the tube. One end of the drainage tube is outside the container through the sealing ring (referred as fluid retention ring hereafter), so the part of the tube outside of the container through the fluid retention ring is not exposed to the solution. It is noted that “fluid retention ring,” as used in the present disclosure, does not necessarily mean a round-shaped object. It can be some other shapes but has to be able to accommodate the cross-section of the tube without a gap. The system of the catheter in the loading solution (in a sealed container) is placed into storage (e.g., a warehouse). As the rubber wall of the tube absorbs the lidocaine dissolved in the solution, lidocaine in the solid form in the solution continues to dissolve into the solution. After sufficient time (typically days or weeks), the wall of the drainage tube has absorbed enough lidocaine for the purpose of extended release of lidocaine. However, in one embodiment, the tube continues to stay in the solution. Eventually, the lidocaine in the solution and that in the wall of the tube reach dynamic equilibrium. The quantity of lidocaine solid initially in the solution can be such that when the dynamic equilibrium is reached, there is no more solid lidocaine in the solution. Optionally, the quantity of lidocaine solid initially in the solution is such that when the dynamic equilibrium is reached, there is no more solid lidocaine in the solution and the concentration of dissolved unionized lidocaine in the solution is at or very close to the saturation point, e.g., is higher than 90% of the solubility of unionized lidocaine in the solution (as measured at 20° C.). When it comes the time to use the drainage tube, the tube is pulled out of the container through the fluid retention ring which “wipes” the solution from the surface of the tube so that the tube pulled out of the container is dry or has only small amount of the solution (less than 1 mm layer) on its wall and can be used as an ordinary “dry” tube. The openings of the tube are unblocked prior to use. Once the drainage tube is in use in the patient, its wall is in contact with the wound surface. Lidocaine in the rubber wall slowly diffuses out of the wall and onto the wound surface. The wound surface is numbed by the continuous presence of lidocaine for an extended period of time. The use of systemic pain control drug may be reduced. Other drugs such as anti-infection agents, anti-inflammatory agents, opioids, etc., can also be embedded into a drainage tube using the same or similar approach.

Many different drug types can be used in accordance with examples of the present disclosure. For examples, as mentioned, local anesthetics can be used. Alternatively or additionally, anti-infection agents such as silver compounds or bases such as chlorhexidine can also or alternatively be embedded into the wall of the medical catheter or tube, such as a drainage tube, using this method. Anti-infection and anti-biofilm formation agents can be embedded into the wall of a medical catheter or tube in accordance with the methods and concepts described above. For example, in one example, a medical device or system can be prepared in accordance with the following process: a Foley catheter with one or both openings blocked off can be placed into a water-based loading solution containing chlorhexidine solid (the chlorhexidine solid can be produced by first dissolving chlorhexidine diacetate and then adding appropriate amount of sodium hydroxide to increase the pH which causes the precipitation of the chlorhexidine solid particles. Alternatively, chlorhexidine diacetate and the pre-calculated amount of NaOH can both be placed into the solution. The NaOH increases the solution pH which can convert ionized chlorhexidine into ununionized form. Other ionized drugs can similarly be modified to unionized form using NaOH or other material, as would be known to one skilled in the art after considering the present disclosure. In this specific example, however, as the rubber wall of the catheter absorbs the chlorhexidine dissolved in the solution, chlorhexidine in the solid form continues to dissolve into the solution. Optionally, the temperature of the solution can be increased to speed up the loading of chlorhexidine into the catheter wall. After a desired amount of chlorhexidine is absorbed into the catheter wall, the catheter can be retrieved from the solution, dried, and stored. When it comes time for use, the catheter can be inserted into the urinary tract of the patient in the same way as an ordinary Foley catheter (e.g., with the help of ordinary lubricant which has no drug-releasing function). Once the catheter is in the urinary tract, chlorhexidine in the rubber wall slowly diffuses out of the wall and comes in contact with the mucosal surface of the subject at the location of the catheter wall. The presence of chlorhexidine on the mucosal surface reduces the potential of infection and biofilm formation. Alternatively or additionally, the catheter can be kept in the loading solution during storage and the viscosity and other properties of the solution (e.g. pH) can be such that the solution can act also as a lubricant jelly. In one example, the catheter and the loading solution/lubricant jelly can be kept in a pouch during storage. At the time of use, the pouch is opened, and the already lubricated catheter can be inserted into the patient's urethral tract.

Although certain systems allow for keeping the catheter or tube in a loading solution pouch until use, this is not required. In some examples, once loading has occurred, the device can be dried before being put into storage. In these cases, sometimes the drug, e.g., lidocaine, in the rubber or silicone wall may diffuse out of the material and present itself as a powder (lidocaine crystals) on the surface of the wall during storage. This is not harmful, but it can reduce and vary the amount of the drug actually stored in the wall, resulting in shortened and variable effective drug release durations. Therefore, comparing the systems of the present disclosure in the various embodiments, the embodiments that utilize a loading solution in which the catheter or tube is submerged during the entire or most of the storage period, and optionally, employing a loading solution that can also work as a lubricant, have the following advantages: (1) It is easier to manufacture as its absorption process (the process of loading the drug into the catheter wall) mostly takes place automatically (may require storage at elevated temperature) instead of utilizing active manufacturing operation. (2) The loaded catheter does not need to be dried during the manufacturing process. (3) The catheter may hold more and more stable amount of the drug in the catheter wall, an advantage provided by the dynamic equilibrium feature of the system. (4) The catheter system is easier to use since the loading solution can also serve as the lubricant so that the physician can save the step of applying lubricant before inserting the catheter into the patient's urinary tract.

In another embodiment, the system is similar to that in the aforementioned embodiments with a lidocaine or other local anesthetic wall concentrations of at least 1 mg/cm² within the catheter wall. Optionally, the lidocaine wall concentration can be at least 3 mg/cm², at least 5 mg/cm², or even at least 8 mg/cm².

In another embodiment, the system is similar to that previously described, with a chlorhexidine wall concentration of at least 0.01 mg/cm². Optionally, the chlorhexidine wall concentration can be at least 0.05 mg/cm², at least 0.1 mg/cm², or even at least 0.2 mg/cm².

In still another embodiment, the system can be similar to that in the aforementioned embodiments, and at least a part of the catheter wall (after dynamic equilibrium is reached) is capable of releasing lidocaine or other local anesthetics continuously at rates of at least 1 mcg/hour/cm², at least 3 mcg/hour/cm², or even at least 5 mcg/hour/cm², for at least 24 hrs when submerged in a pH 7.4 phosphate buffer or a 0.9% sodium chloride-in-water solution.

In another embodiment, at least a part of the catheter wall (after dynamic equilibrium is reached) is capable of releasing lidocaine or other local anesthetics continuously at rates higher than 1 mcg/hour/cm², higher than 3 mcg/hour/cm², or even higher than 5 mcg/hour/cm², for at least 72 hrs when submerged in a pH 7.4 phosphate buffer or 0.9% sodium chloride-in-water solution.

In another embodiment, at least a part of the catheter wall (after dynamic equilibrium is reached) is capable of releasing lidocaine or other local anesthetics continuously at rates higher than 1 mcg/hour/cm², for at least 72 hours, and often at least 240 hrs, when in contact with urinary tract mucosa, e.g., human urinary tract mucosa.

In still another embodiment, the system can be similar to that described in the aforementioned embodiments, and at least a part of the catheter wall (after dynamic equilibrium is reached) is capable of releasing chlorhexidine continuously at rates higher than 0.02 mcg/hour/cm², for at least 24 hrs when submerged in a pH 7.4 phosphate buffer or a 0.9% sodium chloride-in-water solution.

In another embodiment, at least a part of the catheter wall (after dynamic equilibrium is reached) is capable of releasing chlorhexidine continuously at rates higher than 0.02 mcg/hour/cm², or higher than 0.04 mcg/hour/cm², for at least 72 hrs when submerged in a pH 7.4 phosphate buffer or 0.9% sodium chloride-in-water solution.

In another embodiment, at least a part of the catheter wall (after dynamic equilibrium is reached) is capable of releasing chlorhexidine continuously at rates higher than 0.1 mcg/hour/cm² for at least 7 days when submerged in a pH 7.4 phosphate buffer, or 0.9% sodium chloride-in-water solution, or in the urinary tract of a human.

In another embodiment, at least a part of the catheter wall (after dynamic equilibrium is reached) is capable of chlorhexidine continuously at rates higher than 0.1 mcg/hour/cm² for at least 7 days, and often at least 10 days, when in contact with urinary tract mucosa, e.g., human urinary tract mucosa.

It is notable that the local anesthetic agents used in accordance with the present disclosure are typically bases. They have high solubility in water based (aqueous) solutions with low pH, and low solubility in solutions with high pH. For example, lidocaine's solubility in water is higher than 20% when the pH of the water is lower than 5, but the solubility drops to below 0.5% when the pH is higher than 8. Also, the fraction of lidocaine in unionized form, which is much less soluble in water than the ionized molecules, increases with increasing pH. In the present disclosure, the loading solutions used to load local anesthetic agents into the catheter or tube wall have a pH such that most of the local anesthetic agent in the solution exist in the unionized form (mostly undissolved). This approach is counter-intuitive since only the local anesthetic agent dissolved in the loading solution can be absorbed by the rubber wall, an ordinary person skilled in the art would naturally try to maximize the concentration of the drug actually dissolved in the loading solution to maximize the loading speed and quantity. For a base drug such as a lidocaine, this approach would mean using a salt form of the local anesthetic agent (i.e. lidocaine HCl) or other methods to make the pH of the solution significantly below the pKa of the drug. Indeed, almost all commercially available lidocaine solutions have pH lower than about 5.5. (lidocaine's pKa is about 7.9). By using a low pH (e.g. 5.5 or lower), one can easily dissolve more than 20% lidocaine in an aqueous solution. However, in accordance with that disclosed herein, the opposite conclusion is reached. Specifically, it has been discovered that using high solution pH (low solubility) is more effective in loading the local anesthetic into the catheter or tube wall. Some reasons may include the following: Silicone and latex rubbers are hydrophobic materials. Ionized drug molecules, such as most of lidocaine molecules in a solution with a pH much lower than lidocaine's pKa, are hydrophilic. A typical commercially available lidocaine jelly or solution has pH of 5.5 or lower, so practically all the lidocaine molecules in the typical jelly or solution are ionized, dissolved, and hydrophilic. However, hydrophilic substances do not like to permeate into hydrophobic materials. Therefore, even if there is a high concentration of the dissolved drug (i.e. ionized lidocaine) in the solution, the rubber wall will not absorb very much. On the other hand, unionized drug molecules are usually less hydrophilic and thus have better affinity with the rubbers than in the ionized ones, although they usually have much lower (but not zero) water solubility. For example, unionized lidocaine molecules that are dissolved in the loading solution, although less than 0.5%, have much better affinity with silicone or latex rubber than ionized lidocaine molecules do. This factor of better affinity more than offsets the factor of lower concentration, making the high pH loading solutions the better choice than the lower pH loading solutions.

Similar principles apply to drugs other than local anesthetics. For examples, the effect of pH on chlorhexidine loading can be seen in the Examples. Thus methods and systems described herein can use drugs other than, or in addition to, local anesthetics. For example, a silver compound can be used, or an anti-infective (or other similar compound) is also usable, such as chlorhexidine, silver sulfadiazine, triclosan, nitrofurazone, minocycline, rifampicin, ciprofloxacin, fosfomycin, vancomycin, tobramycin, cefamandol, cephalothin, carbenicillin, amoxicillin, gentamicin, flucloxaxillin, ceragenins, flucanazole, furanone, echinocandins, amphotericins, gendine, chitosan, IgG, cephalosporin, ethylenediamine tetraacetic acid (EDTA), metal binding chelators, biofilm inhibiting peptides, and combinations thereof.

The drugs in some of the embodiments of the present disclosure can also include anti-inflammatory agents and opioids. Anti-inflammatory agents include, without limitation, non-steroidal anti-inflammatory drugs (NSAIDS) such as ketoprofen, diclofenac, Ibuprofen, Indomethacin, salicylates, mefenamic acid, meloxicam, nabumetone, naproxen, oxaprozin, piroxicam, rofecoxib, sulindac, valdecoxib, and their salts. Anti-inflammatory agents can also include certain steroids such as hydrocortisone, hydrocortisone acetate, cortisone acetate, tixocortol pivalate, prednisolone, methylprednisolone, prednisone, Triamcinolone acetonide, triamcinolone alcohol, mometasone, amcinonide, budesonide, desonide, fluocinonide, fluocinolone acetonide, halcinonide, betamethasone sodium phosphate, dexamethasone, dexamethasone sodium phosphate, fluocortolone, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate, fluprednidene acetate, h, 17-aceponate, 17-buteprate, and prednicarbate.

Opioids in some embodiments of the present disclosure can include, without limitation, cocaine, morphine, fentanyl, miperidine, oxycodone, hydrocodone, codeine, hydromorphone, buprenorphine, methadone, sufentanil, remifentanil, and tramadol. While some opioids, such as cocaine, can produce a local anesthetic effect, the main acting site of opioids is in the brain. Therefore, controlled extended release of an opioid drug from a catheter or tube surface on a patient's wound or mucoal surface releases the drug into the patient's systemic circulation, and the drug eventually enters the brain, in a controlled and extended fashion.

When a medical catheter or tube's rubber wall is submerged into a loading solution with a pH high enough such that most of the lidocaine molecules are unionized, the rubber wall absorbs the dissolved unionzed lidocaine at high rates, despite that unionized lidocaine's concentration is very low (limited by the low solubility of the unionized species). As the dissolved unionzed lidocaine leaves the solution and enters the rubber wall, undissolved lidocaine continues to dissolve into the solution and maintains the concentration of the dissolved unionzed lidocaine at saturation. If the process is allowed to continue, the lidocaine in the rubber wall and that in the solution will eventually reach dynamic equilibrium. At that point, if the loading solution initially contained sufficient amount of lidocaine (even if most of it is in undissolved form), the rubber wall will contain much more drug than what a lower pH solution can load.

The above illustration also applies to drugs other than lidocaine. A factor to consider with other drugs is to use a loading solution pH such that most of the drug in the load solution initially is unionized. For example, chlorhexidine is a base, and as an active ingredient, is often supplied in the form of chlorhexidine diacetate and chlorhexidine gluconate. Chlorhexidine diacetate can be dissolved in water with concentrations higher than 1.5 wt % to produce a solution with pH about 7.4. If the pH is increased by adding a base such as NaOH, the number of unionized molecules should increase. As a result, more chlorhexidine can be loaded into the catheter wall. Indeed, this was observed in a subsequent example hereinafter (Example 10). Similar principles would also be observable with other drugs that can be modified to a more ionized form to a more unionized form.

In another embodiment, a drug-embedded medical catheter or tube can be stored (during shelf life) as follows. At least a part of the medical catheter or tube already embedded with a drug can be kept in a solution containing the same drug for most of or the entire period from initial preparation and storage until the product is used. Often, the storage solution will also be the loading solution. If a drug embedded medical catheter tube is stored in a pouch by itself, the embedded drug may or may not migrate out of the wall material of the catheter or tube, depending on factors such as the surface concentration of the embedded drug, or the temperature and moisture level exposure. Storing the drug-embedded catheter or tube in the solution containing the same drug can minimize or eliminate the out-migration potential of the drug, which is driven in part by the drug concentration difference between inside and outside of the wall of the catheter or tube. The concentration of the drug in the solution reduces or eliminates this concentration difference and thus the out-migration potential.

In the embodiments of the present disclosure, the drug can be absorbed or loaded into the wall material of the catheter or tube instead of in an additional layer of material coated on the wall surface for the purpose of attaching the drug to the catheter or tube. This is a specific distinction between the embodiments of the present disclosure and other approaches which merely coat a layer of material containing the drug onto the surface of the walls of these types of devices.

The aforementioned embodiments provide applications of more general methods and systems of the present disclosure for loading a drug or drugs into the a medical device. This can be specifically carried out by submerging a surface of a medical device into a solution comprising a drug or multiple drugs for a period of time for the drug in the solution to be absorbed into the wall of the catheter.

In short, a minimum length of the time for this loading process can be dependent on the temperature of the solution and the catheter. It can be as short as less than one hour at certain temperatures, e.g., greater than or equal to about 100° C., and can be many hours or days at other temperatures, e.g., 50° C. to 70° C. It can even be greater than 15 or 30 days if the temperature is lower, e.g., room temperature, 35° C., 40° C. After the drug is loaded into the wall of the catheter, the catheter can, optionally, stay in the solution until it is used.

### EXAMPLES

The following examples illustrate the embodiments of the disclosure. However, it is to be understood that the following are only exemplary or illustrative of the application of the principles of the present disclosure. Numerous modifications and alternative compositions, methods, and systems may be devised by those skilled in the art without departing from the spirit and scope of the present disclosure. The appended claims are intended to cover such modifications and arrangements. Thus, while the present disclosure has been described above with particularity, the following examples provide further detail in connection with what are presently deemed to be practical embodiments of the disclosure.

### Example 1

### Lidocaine Loading Method (Foley Catheter)

TABLE 1
Foley Catheter Used

| | Catheter A | Catheter B |
|---|---|---|
| Material | Silicone Coated | All Silicone |
| | Latex | |
| Size (French Units) | 16 F | 16 F |
| Brand, Model | Bardia | Bardex |
| | 123516a | 165816 |
| Manufacturer | CR Bard | CR Bard |
| Lot No. | Myubr246 | Ngub2781 |

### Lidocaine Loading Process

Catheter A

400 mg of lidocaine base was placed in a 30 mL glass bottle with 18 mL of a blank loading solution (11.5 wt % glycerin, 1.5 wt % Natrasol H, NF, 0.1 wt % methyl paraben, 86.9 wt % water). This solution can also function as lubricant jelly. The Foley catheter, with all openings blocked, was placed into the solution and completely submerged in a spiral shape. The bottle was then placed into a Styrofoam® box and heated in an oven with digital temperature control. The temperature inside the box was maintained at about 40-41° C. for the next 48 days. The bottle was then removed from the oven and stored at room temperature (for about 9 months) until the lidocaine release tests set forth in Example 2 below.

Most of the 400 mg lidocaine base did not dissolve in the solution initially and stayed as crystals at the bottom of the glass bottle. However, the amount of undissolved lidocaine crystals continued to decrease slowly during the 48 day high temperature storage and completely disappeared at about the end of the 48 day period. Observations were recorded at days 1, 10, 20, 37, 49, and 80 (from the bottom view of glass bottle) to monitor lidocaine disappearance as a function of time.

Specifically, the lidocaine crystals were observed to diminish over time until they were observed to be completely gone at observation day 49. Calculations using the lidocaine concentration that remained in the loading solution (about 4 mg/mL, dissolved lidocaine) indicated that about 328 mg of the lidocaine was absorbed into the catheter. The total surface area of this catheter was about 80 cm², so the average amount of lidocaine embedded in each square centimeter was about 4 mg.

Catheter B

The same lidocaine loading process was used to load Catheter B as was used for Catheter A, except that the 40-41° C. loading/heating period was observed for only 37 days instead of about 48 days. Observations were recorded at days 2, 9, 16, 26, 38 (start of room temperature storage), and 69. Specifically, the lidocaine crystals were observed to diminish over time until about day 26, and then the crystals remained constant until heating stopped through day 37. The amount of lidocaine crystals stayed about the same during room temperature storage between Day 38 and Day 69 (and next 8 months), suggesting the dynamic equilibrium of lidocaine between the catheter wall and the solution was reached by Day 26. Because there was undissolved lidocaine in the solution at the equilibrium, the solution had a saturated lidocaine concentration.

### Example 2

### In Vitro Release Experimental Results

Catheters A and B were used as samples for the in vitro lidocaine release test as follows:

Lidocaine Release Test Procedure

Foley catheters A and B were taken out of their loading solutions and rinsed with running water for 30 seconds to remove any particles from their respective surfaces. The catheter surfaces were dried with a paper tissue. The catheter was then wiped with an ethanol soaked tissue paper to remove possible lidocaine on the surface and dried with a tissue paper (this would not be required for patient application, but is useful in this example to show drug release from within the wall of the catheter). The Foley catheters were then cut into 6 pieces: the tip, the balloon, the discharge end, and three approximate 10 cm equal parts (sections B, C, and D) of the tube body between the balloon and the discharge end. The tip and the discharge end were discarded because they would not be expected to touch the patient's mucosa during catheter use. The balloon piece and the three equal tube body parts were used for the in vitro lidocaine release measurements set forth in Tables 2A and 2B below. The holes on both ends of each of the 4 catheter sections were blocked so only lidocaine released from the outer wall was measured. Each of the 4 catheter sections were placed into a glass scintillation vials. A 0.15 M PBS (Phosphate buffer solution) solution with pH 7.4 was used as release medium. Twenty and seven tenth of the medium was added into the vial to submerge each entire catheter section. The vial was closed tightly and a vinyl tape was wrapped around the cap to prevent solvent evaporation. The vial was placed into a 37° C. water bath with no shaking, and the time was set as t=0. At each of the specified time points for the next 14 days, a sample was taken from the solution in each of the sample vials, and the catheter sections were removed from the vial, dried with a tissue, and placed into a new vial with the same amount of release medium. The new vial was then put back into the same water bath. This process was repeated at each time point. The samples were analyzed with HPLC, and the amount of lidocaine released during each time period (between the current and previous time points) was determined. In addition, lidocaine concentrations in the loading solutions that came with the samples were also analyzed by HPLC with proper dilution.

In Vitro Lidocaine Release Results

The following tables (2A and 2B) list the calculated lidocaine release flux, in microgram per square centimeter per hour (mcg/cm²/hr), from the two catheters over the 14-day test period.

TABLE 2A
Catheter A (silicone coated latex) Flux (mcg/cm²/hr)

| Time | Time | Balloon | Section B | Section C | Section D |
|---|---|---|---|---|---|
| (hours) | (days) | Flux | flux | flux | flux |
| 4 | 0.17 | 124.1 | 121.2 | 115.9 | 108.2 |
| 12 | 0.5 | 58.2 | 61.6 | 54.3 | 48.4 |
| 24 | 1 | 36.2 | 42.7 | 36.1 | 31.2 |
| 36 | 1.5 | 26.5 | 33.3 | 28.7 | 24.9 |
| 48 | 2 | 22.1 | 28.4 | 23.3 | 19.8 |
| 61 | 2.5 | 17.1 | 21.1 | 17.7 | 15.5 |
| 72 | 3 | 16.1 | 19.9 | 16.2 | 13.6 |
| 84 | 3.5 | 13.2 | 15.4 | 12.5 | 10.6 |
| 96 | 4 | 11.3 | 13.0 | 10.3 | 8.7 |
| 108 | 4.5 | 9.7 | 10.6 | 8.3 | 7.3 |
| 120 | 5 | 8.4 | 9.3 | 7.1 | 6.1 |
| 132 | 5.5 | 7.2 | 7.3 | 6.0 | 5.4 |
| 144 | 6 | 6.3 | 6.4 | 5.4 | 4.5 |
| 168 | 7 | 4.2 | 4.3 | 3.3 | 2.8 |
| 180 | 7.5 | 4.3 | 4.4 | 3.3 | 2.8 |
| 192 | 8 | 3.5 | 3.5 | 2.5 | 2.1 |
| 204 | 8.5 | 3.2 | 3.0 | 2.2 | 1.8 |
| 216 | 9 | 2.5 | 2.4 | 1.8 | 1.5 |
| 228 | 9.5 | 2.0 | 2.0 | 1.5 | 1.2 |
| 240 | 10 | 1.8 | 1.7 | 1.2 | 1.0 |
| 252 | 10.5 | 1.5 | 1.4 | 1.0 | 0.8 |
| 265.5 | 11.1 | 1.4 | 1.2 | 0.9 | 0.7 |
| 276 | 11.5 | 1.2 | 1.1 | 0.9 | 0.7 |
| 288 | 12 | 1.1 | 1.0 | 0.7 | 0.5 |
| 300 | 12.5 | 0.9 | 0.8 | 0.6 | 0.4 |
| 312.5 | 13. | 0.7 | 0.7 | 0.5 | 0.4 |
| 324 | 13.5 | 0.7 | 0.6 | 0.5 | 0.4 |
| 336 | 14 | 0.6 | 0.5 | 0.4 | 0.3 |

TABLE 2B
Catheter B (all silicone) Flux (mcg/cm²/hr)

| Time | Time | Balloon | Section B | Section C | Section D |
|---|---|---|---|---|---|
| (hours) | (days) | Flux | flux | flux | flux |
| 4 | 0.17 | 259.3 | 208.8 | 203.7 | 200.7 |
| 12 | 0.5 | 78.5 | 55.5 | 51.5 | 49.4 |
| 24 | 1 | 34.1 | 18.3 | 16 | 16 |
| 36 | 1.5 | 16.3 | 8.6 | 7.2 | 7.4 |
| 48 | 2 | 9.0 | 4.9 | 4.4 | 4.3 |
| 61 | 2.5 | 5.4 | 3.0 | 2.4 | 2.4 |
| 72 | 3 | 4.8 | 2.7 | 2.0 | 2.4 |
| 84 | 3.5 | 3.2 | 1.9 | 1.6 | 1.7 |
| 96 | 4 | 2.6 | 1.8 | 1.4 | 1.4 |
| 108 | 4.5 | 2.3 | 1.3 | 1.0 | 1.3 |
| 120 | 5 | 1.9 | 1.2 | 1.0 | 1.0 |
| 132 | 5.5 | 1.9 | 1.4 | 1.1 | 1.0 |
| 144 | 6 | 1.9 | 1.2 | 1.0 | 1.0 |
| 168 | 7 | 0.9 | 0.5 | 0.4 | 0.7 |
| 180 | 7.5 | 1.4 | 0.8 | 0.7 | 0.7 |
| 192 | 8 | 1.1 | 0.7 | 0.6 | 0.6 |
| 204 | 8.5 | 1.0 | 0.6 | 0.5 | 0.5 |
| 216 | 9 | 0.9 | 0.5 | 0.5 | 0.5 |
| 228 | 9.5 | 0.7 | 0.5 | 0.4 | 0.4 |
| 240 | 10 | 0.8 | 0.4 | 0.4 | 0.4 |
| 252 | 10.5 | 0.8 | 0.4 | 0.4 | 0.4 |
| 265.5 | 11.1 | 0.7 | 0.4 | 0.4 | 0.3 |
| 276 | 11.5 | 0.8 | 0.5 | 0.4 | 0.4 |
| 288 | 12 | 0.6 | 0.4 | 0.4 | 0.3 |
| 300 | 12.5 | 0.6 | 0.3 | 0.3 | 0.3 |
| 312.5 | 13 | 0.6 | 0.3 | 0.3 | 0.3 |
| 324 | 13.5 | 0.6 | 0.3 | 0.3 | 0.3 |
| 336 | 14 | 0.5 | 0.3 | 0.3 | 0.3 |

For catheters A and B, the lidocaine release flux stayed above 1 mcg/cm²/hr for more than 10 days and 6 days, respectively. For catheter A, lidocaine release fluxes out of Sections B, C, and D are in decreasing order at most of the time points. That is likely due to the fact that the loading solution was not saturated (equilibrium lidocaine concentration in the loading solution for Catheter A was 4 mg/mL, lower than the 4.6 mg/mL saturation concentration), so the loading solution closer to the lidocaine crystals sitting at the bottom of the glass bottle had higher lidocaine concentration. As a result, the sections closer to the bottom of the glass bottle were exposed to higher lidocaine concentrations and thus absorbed more lidocaine. The balloon section was the closest to the undissolved lidocaine particles, followed by sections B, C, and D. Lidocaine fluxes out of the different sections of Catheter B were more even, likely due to the fact that the loading solution for Catheter B had saturated lidocaine concentration so that the lidocaine concentration was the same essentially everywhere in the loading solution. Furthermore, it is noted that even the peak flux is extremely safe. The highest flux (259.3 mcg/cm²/hr, at hour 4, balloon section, Catheter B) is equivalent to infusing about 1.5 mL of a 1 wt % lidocaine solution into the patient per hour.

The above results suggest that hundreds of mg of lidocaine can be embedded into the wall of the catheter using the methods described herein. However, at room temperature, the process can take more than a month. Heating can increase the loading speed of these devices.

### Example 3

### Simultaneously Diffusing a Drug into the Wall of a Foley Catheter and Sterilizing the Catheter with High Temperature

Diffusing sufficient amount of lidocaine or another drug into the wall of a catheter, such as Foley catheter, at room temperature or even slightly higher temperatures can take weeks to months. For example, the process of absorbing about 328 mg of lidocaine into Catheter A (Example 1) at a slightly elevated temperature (40-41° C.) took about 38 days. A Foley catheter for medical use can benefit from sterilization, and sterilization by ethylene is an industry standard method, requiring an additional unnecessary step. In this example, a Foley catheter in a lidocaine loading solution can be sterilized with high temperature and if the catheter can absorb sufficient amount of lidocaine during the autoclaving process. Three separate Experiments were conducted (A, B, and C), as described below.

### Experiment A

Specifically, 550 mg of lidocaine base was placed into a 30 mL glass bottle. A silicone coated latex Foley catheter (Bardia brand, model 123516A, from CR Bard), with all openings blocked, was placed in the bottle with the lidocaine base. 18 mL of a blank loading solution (1.5 wt % Natrosol 250H NF, 0.1 wt % methyl paraben, and 98.4 wt % water) was then placed into the glass bottle. Most of the lidocaine particles stayed dissolved in the solution, as designed. The bottle was closed with a metal cap with plastic seal. The closed bottle was placed into an electric pressure cooker (Cuisinard brand, Model CPC-600) with pressure set at “high” and time set at 30 minutes. (According to Cuisinart, the pressure inside the cooker should be 10 PSI which indicates a temperature of about 115° C.). The entire heating process (including time to reach the set pressure, the 20 min “cooking time”, and the time for the pressure inside the cooker to be low enough so that the cooker could be opened) took about one hour. After the heating process, the bottle was removed from the cooker and let cool to room temperature. All lidocaine particles in the solution had disappeared and the solution was clear, suggesting that the initially undissolved lidocaine particles had been absorbed by the catheter. The catheter was removed from the bottle, washed with water and dried. 5 mL of water was injected into the balloon via the injection port to inflate the balloon. The balloon was inflated without any problem and stayed inflated for at least the next 6 days.

### Experiment B

690 mg of lidocaine base was placed into a 4 mil thick polypropylene bag (4 inch×6 inch, S-13265, Uline). Forty gram of a blank loading solution (1.5 wt % Natrosol 250H NF, 11.5 wt % glycerin, 0.1 wt % methyl paraben, and 86.9 wt % water) was also placed into the bag. A silicone coated latex Foley Catheter (Bardia brand, model 123516A, from CR Bard), with all openings blocked, was placed into the bag. After most of the air in the bag was squeezed out, the bag was sealed with an impulse sealer. The bag was massaged to make the undissolved lidocaine particles distribute more evenly. The bag was placed into the same electric pressure cooker with pressure set at “high” and time set at 20 minutes. The entire heating process took about 60 minutes. After the heating process was complete, the bag was removed from the cooker and allowed to cool to room temperature. All initially undissolved lidocaine particles inside the bag had disappeared, suggesting that they had all been absorbed by the catheter.

### Experiment C

Two glass vials, each containing about 0.15 g of lidocaine in 6 mL water were treated with a normal autoclave process. Lidocaine contents in the vials were measured with an HPLC method. The recovery of lidocaine in the two vials was found to be 98.3 wt % and 99.5 wt %, respectively, which are well within the experimental error range.

Discussion

The above experimental results (Experiments A, B, and C) suggest that (1) the Foley catheter can withstand the temperature and pressure of autoclave process without damage. (2) The autoclave temperature and pressure does not cause significant degradation to lidocaine in water (and thus so for lidocaine in water based loading solutions). (3) Therapeutically sufficient amount of lidocaine in the loading solution (the initially undissolved particles, about 470 mg out of 550 mg in Experiment 1, and 510 mg out of 690 mg in Experiment 2) can be absorbed into the wall of the catheter in a typical autoclave process. This finding was surprising because it takes about 40 days for the same catheter to absorb about 328 mg lidocaine at 40-41° C. (Example 1). Although it might have been expected that higher temperature would make the absorption process faster, the higher temperature was not expected to shorten the absorption process from about 40 days to no more than 60 minutes. This quick absorption implies that the lidocaine loading and autoclave sterilization can be accomplished in the same short process.

### Example 4

### Chlorhexidine Loading Method (Foley Catheter)

0.16 g of chlorhexidine diacetate was dissolved in 15.8 gram distilled water in a glass jar to form a clear solution. A section of a silicone coated latex Foley catheter (Bardia Foley Catheter, 16 Fr. Reorder No. 123516A, Lot MYUDR067), approximately 16 cm long, was placed into the jar and completely submerged in the solution. 0.08 g of a 10 wt % NaOH solution was added into the loading solution to increase the pH. Immediately after the addition of the NaOH solution, the loading solution in the jar became milky white, indicating that a significant amount of the chlorhexidine had precipitated out of the solution because its solubility had been significantly reduced by the NaOH. The jar was closed with a cap and placed into an oven with temperatures cycling between about 62° C. and 68° C. The jar was kept in the oven for the next 120 hours and was shaken occasionally during the 120 hour heat treatment period.

It was observed that the amount of undissolved chlorhexidine decreased gradually during the heat treatment period, and after 96 hours of heating, the solution became clear with no visible solid particles. After the 120 hour heat treatment, the jar was removed from the oven and kept at room temperature for the next about four months before it was used for the chlorhexidine release experiment described below in Example 5. It was observed that the solution stayed almost clear (except for some very small amount of solid particles, likely chlorhexidine, observed at the bottom of the jar) during the room temperature storage period.

### Example 5

### In Vitro Release Experimental Results

The release rates of chlorhexidine from the loaded catheter in Example 4 were measured as follows. The catheter section was thoroughly washed and cut into two pieces, Section A (7.774 cm) and Section B (8.055 cm). Each section was submerged into 17 mL of a 0.15 M pH7.4 phosphate buffer held in a glass vial, which was placed into a 37° C. water bath. At each time point, the catheter section was retrieved from the glass vial, dried, and placed into another glass vial containing 17 mL of a fresh pH 7.4 phosphate buffer. The vial was then placed into the 37° C. water bath. The amount of chlorhexidine in the solution remaining in the first vial was measured with HPLC. This process was repeated at each time point. The average flux of chlorhexidine released from the catheter wall during the period between the current and previous time points was calculated by dividing the total amount of chlorhexidine in the solution (microgram) by the total surface area of the catheter section (cm²) and by the length of time between the two time points (hour). The result is the amount of chlorhexidine released per square centimeter of the catheter wall per hour. The average chlorhexidine release flux and the average amount of chlorhexidine released from each square centimeter of the catheter wall per 24 hour period are listed in Table 3.

TABLE 3
Flux of Chlorhexidine over 14 Day Period

| | Average | |
|---|---|---|
| | Chlorhexidine | Average |
| | Flux for Sections | Chlorhexidine |
| Time | A and B | Amount/cm²/24 hr |
| (day) | (mcg/hr/cm²) | (micrograms) |
| 0.17 | 9.08 | 218 |
| 0.5 | 1.83 | 43.9 |
| 1.1 | 0.89 | 21.5 |
| 1.6 | 0.55 | 13.1 |
| 2 | 0.42 | 10.2 |
| 2.5 | 0.19 | 4.5 |
| 3 | 0.18 | 4.3 |
| 3.5 | 0.11 | 2.6 |
| 4.0 | 0.14 | 3.3 |
| 4.5 | 0.14 | 3.2 |
| 5 | 0.13 | 3.2 |
| 5.5 | 0.12 | 2.9 |
| 6 | 0.11 | 2.6 |
| 6.5 | 0.09 | 2.2 |
| 7 | 0.07 | 1.8 |
| 8 | 0.05 | 1.2 |
| 8.5 | 0.06 | 1.4 |
| 9.0 | 0.07 | 1.7 |
| 10.0 | 0.08 | 2.0 |
| 11 | 0.05 | 1.1 |
| 12 | 0.03 | 0.8 |

The above results indicate that enough chlorhexidine can be embedded into the wall of a catheter so that more than 1 microgram of chlorhexidine can be released from each square centimeter of the wall surface per 24 hour period for at least 11 days.

### Example 6

### In Vitro Simultaneous Loading of Lidocaine and Chlorhexidine (Foley Catheter)

A silicone coated latex Foley catheter (size 16 F, Bardia, 123516a, CR Bard) was simultaneously loaded with Lidocaine and Chlorhexidine. The catheter, with openings blocked, was submerged in a solution of 18 g distilled water, 0.61 g lidocaine base, and 0.54 g chlorhexidine diacetate. The catheter and the solution, in a glass jar, were heated at 122° F. (50° C.) for 48 hours.

### Example 7

### Extended Release of Lidocaine and Chlorhexidine (Foley Catheter)

The lidocaine loaded Foley catheter of Example 6 was cut into six pieces: the tip, the balloon, the discharge end, and three 10 cm equal parts (sections B, C, and D) of the tube body between the balloon and the discharge end. The tip and the discharge end were discarded because they would not be expected to touch the patient's mucosa during the catheter use. The balloon section and the three equal tube body sections were used for the in vitro lidocaine release measurement. The holes on both ends of each of the four catheter sections were blocked. Each of the four catheter sections was placed into a glass scintillation vial. A 0.15 M PBS solution with pH 7.4 was used as release medium. 20.7 mL of the medium was added into the vial to submerge the entire catheter section. The vial was closed tightly and a vinyl tape was wrapped around the cap to prevent solvent evaporation. The vial was placed into a 37° C. water bath with no shaking (mimicking human use), and the time was set as t=0. At each of the specified time points for the next 14 days, a sample was taken from the solution in each of the sample vials. The catheter sections was removed from the vial, dried with a tissue, and placed into a new vial with the same amount of release medium. The new vial was then put back into the same water bath. This process was repeated at each time point. The samples were analyzed with HPLC. The amounts of lidocaine and chlorhexidine released during each time period (between the current and previous time points) were determined.

TABLE 4
Flux and Drug Release Data for
Chlorhexidine and Lidocaine over 14 days

| | | | Average |
|---|---|---|---|
| | Average Llidocaine | Average | Released |
| | Flux for Sections | Chlorhexidine Flux for | Chlorhexidine |
| Time | B, C, and D | Sections B, C, and D | Amount/cm²/24 hr |
| (day) | (mcg/hr/cm²) | (mcg/hr/cm²) | (micrograms) |
| 0.17 | 213 | 0.09 | 2.2 |
| 0.5 | 64 | 0.16 | 3.8 |
| 1.0 | 51 | 0.12 | 2.8 |
| 1.5 | 43 | 0.15 | 3.4 |
| 2 | 38 | 0.13 | 3.2 |
| 2.5 | 30 | 0.10 | 2.4 |
| 3.1 | 22 | 0.07 | 1.6 |
| 3.6 | 20 | 0.06 | 1.5 |
| 4 | 18 | 0.07 | 1.7 |
| 4.5 | 14 | 0.04 | 1.0 |
| 5 | 12 | 0.05 | 1.3 |
| 5.5 | 11 | 0.04 | 1.0 |
| 6.5 | 6.7 | 0.03 | 0.6 |
| 7 | 7.2 | 0.03 | 0.7 |
| 7.5 | 6.1 | 0.03 | 0.6 |
| 8 | 4.5 | 0.02 | 0.5 |
| 9 | 3.0 | 0.02 | 0.5 |
| 9.5 | 2.8 | 0.03 | 0.5 |
| 10 | 2.2 | 0.02 | 0.5 |
| 11 | 1.5 | 0.02 | 0.5 |
| 12 | 1.1 | 0.02 | 0.5 |
| 14 | 0.6 | 0.02 | 0.4 |

The above results suggest that lidocaine and chlorhexidine can be loaded into the catheter wall and released from the catheter wall simultaneously. The lidocaine release flux in this experiment is generally higher than that in Example 1. It was unexpected that a mere 10° C. temperature increase (from 40° C. to 50° C.) can shorten the lidocaine loading time from about 40 days to about 2 days.

### Example 8

### Use of a Foley Catheter Embedded with Lidocaine

A Foley catheter is embedded with lidocaine, similar to that in Experiment A of Example 3, is provided to a caregiver. The catheter is taken out of the sealed pouch and a lubricant, commonly used in the procedure, is smeared on the surface. The catheter is then inserted into the patient's urinary tract. Once it is in place, the surface of the catheter is in contact with the mucosa of the urinary tract. Lidocaine in the catheter wall then slowly permeates out of the wall and enters the mucosa, resulting in extended release of lidocaine from the wall. The patient's mucosa is numbed for an extended period of time (e.g. more than 72 hours).

### Example 9

### Use of a Central Venous Catheter Embedded with Chlorhexidine

A central venous catheter embedded with the anti-infection agent chlorhexidine is manufactured using a method similar to that used in Example 4 (using a central venous catheter rather than a Foley catheter). The catheter can be used with decreased risk of infection by a patient.

### Example 10

### Effect of pH on Embedding of Drugs and Release Rates

Three identical Foley catheters were embedded with lidocaine and chlorhexidine under different conditions, mainly different solution pHs. Specifically, the catheters were silicone coated latex catheters (size 16 F, Bardia 123516A, CR Bard). Each of the catheters was submerged in a loading solution held in a 30 mL glass jar. The jars were heated in a 122° F. (50° C.) oven for 48 hours (catheters 1 and 2) or 96 hours (catheter 3). The solution in each jar, about 18 mL in volume, contained 0.54 g chlorhexidine diacetate and 0.61 g lidocaine base. Both drugs initially mostly existed as undissolved particles in the solution. Variable amounts of 5% NaOH, which is used to increase the solution pH, were included, as listed in Table 5.

TABLE 5
pH of Loading Solutions

| | Amount of 5% | pH of the loading solution |
|---|---|---|
| | NaOH | at the time of release test |
| Catheter 1 | 0 | 7.4 |
| Catheter 2 | 0.15 g | 8.1 |
| Catheter 3 | 0.44 g | 8.7 |

The release profiles of chlorhexidine and lidocaine from each of the catheters was measured over about a 14 day period with a method similar to that used in Example 7. It should be noted that all quantities of chlorhexidine are expressed in the equivalence of chlorhexidine diacetate which was used to make HPLC standards. Table 6 below lists the mean chlorhexidine flux (averages of sections B, C, and D) for each catheter. Table 7 below lists the mean lidocaine flux (averages of sections B, C, and D) for each catheter as well.

TABLE 6
Chlorhexidine Flux

| Catheter 1 | | Catheter 2 | | Catheter 3 | |
|---|---|---|---|---|---|
| | Mean | | Mean | | Mean |
| | Chlorhexidine | | Chlorhexidine | | Chlorhexidine |
| Time | Flux | Time | Flux | Time | Flux |
| (day) | (mcg/hr/cm²) | (day) | (mcg/hr/cm²) | (day) | (mcg/hr/cm²) |
| 0.17 | 0.09 | 0.17 | 0.96 | 0.17 | 16.61 |
| 0.5 | 0.16 | 0.5 | 0.20 | 0.5 | 2.88 |
| 1.0 | 0.12 | 1.04 | 0.18 | 1.04 | 0.93 |
| 1.5 | 0.15 | 1.5 | 0.08 | 1.5 | 0.56 |
| 2 | 0.13 | 2 | 0.06 | 2 | 0.38 |
| 2.5 | 0.10 | 2.52 | 0.06 | 2.52 | 0.37 |
| 3.1 | 0.07 | 3 | 0.04 | 3 | 0.30 |
| 3.6 | 0.06 | 3.5 | 0.05 | 3.5 | 0.26 |
| 4 | 0.07 | 4.02 | 0.05 | 4.02 | 0.24 |
| 4.5 | 0.04 | 4.5 | 0.05 | 4.5 | 0.29 |
| 5 | 0.05 | 5.02 | 0.04 | 5.02 | 0.27 |
| 5.5 | 0.04 | 5.5 | 0.04 | 5.5 | 0.27 |
| 6.5 | 0.03 | 6 | 0.03 | 6 | 0.20 |
| 7 | 0.03 | 7.04 | 0.03 | 7.04 | 0.25 |
| 7.5 | 0.03 | 7.52 | 0.04 | 7.52 | 0.25 |
| 8 | 0.02 | 8.02 | 0.04 | 8.02 | 0.22 |
| 9 | 0.02 | 9 | 0.03 | 9 | 0.22 |
| 9.5 | 0.03 | 9.5 | 0.03 | 9.5 | 0.25 |
| 10 | 0.02 | 10 | 0.03 | 10 | 0.18 |
| 11 | 0.02 | 11 | 0.04 | 11 | 0.19 |
| 12 | 0.02 | 12.04 | 0.03 | 12.04 | 0.17 |
| 14 | 0.02 | 13.08 | 0.03 | 13.08 | 0.16 |
| 14.25 | — | 14.25 | 0.03 | 14.25 | 0.14 |

The average amount of chlorhexidine released from each cm² of catheter wall (sections B, C, and D) during the 14 days was calculated as 16.2 mcg for Catheter 1, 20.1 mcg for Catheter 2, and 101.55 mcg for catheter 3, indicating that the average quantities of chlorhexidine embedded into each square centimeter of the catheter walls were higher than those quantities.

It can be seen that Catheter 3, whose loading solution had higher pH and whose heat treatment was longer, released much more chlorhexidine than Catheters 1 and 2 over the 14 days. Catheter 3's dramatically higher release rates indicate that higher pH (even by a mere 0.6 and 1.3 units, respectively) can embed significantly more chlorhexidine into the catheter. Although the length of time for the heat treatment of Catheter 3 was 96 hours, and that for Catheters 1 and 2 was only 48 hours, it is believed that the higher drug load in Catheter 3 was mainly due to the higher pH, because the release rates were far more than just doubled.

TABLE 7
Lidocaine Flux

| Catheter 1 | | Catheter 2 | | Catheter 3 | |
|---|---|---|---|---|---|
| | Mean | | Mean | | Mean |
| | Lidocaine | | Lidocaine | | Lidocaine |
| Time | Flux | Time | Flux | Time | Flux |
| (day) | (mcg/hr/cm²) | (day) | (mcg/hr/cm²) | (day) | (mcg/hr/cm²) |
| 0.17 | 213.50 | 0.17 | 160.53 | 0.17 | 131.34 |
| 0.5 | 64.41 | 0.5 | 75.09 | 0.5 | 69.02 |
| 1.0 | 50.93 | 1.04 | 40.53 | 1.04 | 40.15 |
| 1.5 | 42.74 | 1.5 | 36.63 | 1.5 | 35.15 |
| 2 | 38.49 | 2 | 27.41 | 2 | 31.14 |
| 2.5 | 29.82 | 2.52 | 22.51 | 2.52 | 23.57 |
| 3.1 | 22.33 | 3 | 19.63 | 3 | 20.43 |
| 3.6 | 20.14 | 3.5 | 15.79 | 3.5 | 16.50 |
| 4 | 18.49 | 4.02 | 13.46 | 4.02 | 14.00 |
| 4.5 | 13.73 | 4.5 | 11.61 | 4.5 | 11.42 |
| 5 | 12.37 | 5.02 | 9.50 | 5.02 | 9.21 |
| 5.5 | 10.87 | 5.5 | 8.31 | 5.5 | 8.17 |
| 6.5 | 6.74 | 6 | 6.76 | 6 | 5.60 |
| 7 | 7.21 | 7.04 | 4.16 | 7.04 | 4.65 |
| 7.5 | 6.09 | 7.52 | 4.86 | 7.52 | 4.79 |
| 8 | 4.50 | 8.02 | 3.61 | 8.02 | 3.61 |
| 9 | 2.96 | 9 | 2.44 | 9 | 2.43 |
| 9.5 | 2.82 | 9.5 | 2.52 | 9.5 | 2.57 |
| 10 | 2.15 | 10 | 1.85 | 10 | 1.92 |
| 11 | 1.46 | 11 | 1.34 | 11 | 1.31 |
| 12 | 1.09 | 12.04 | 1.08 | 12.04 | 1.05 |
| 14 | 0.63 | 13.08 | 0.79 | 13.08 | 0.79 |
| 14.25 | — | 14.25 | 0.56 | 14.25 | 0.56 |

The average amount of lidocaine released from each cm² of catheter wall (sections B, C, and D) during the 14 days was 5108 mcg for Catheter 1, 4203 mcg for catheter 2, and 4093 mcg for Catheter 3.

While the disclosure has been described with reference to certain embodiments, those skilled in the art will appreciate that various modifications, changes, omissions, and substitutions can be made without departing from the spirit of the disclosure. It is therefore intended that the disclosure be limited only by the scope of the appended claims.

1. A method for embedding a drug into a wall of a medical catheter or tube for sustained release delivery, comprising
submerging a medical catheter or tube into a loading solution including a drug, and
placing the medical catheter or tube and the loading solution into an environment having a temperature of at least 35° C., wherein the drug becomes embedded in a wall of the medical catheter or tube via diffusion from the loading solution into the wall.

2. The method of claim 1, wherein the temperature is at least 40° C.

3. The method of claim 2, wherein the medical catheter or tube is exposed to the temperature for at least 7 days.

4. The method of claim 1, wherein the temperature is at least 50° C.

5. The method of claim 4, wherein the medical catheter or tube is exposed to the temperature for at least 12 hours.

6. (canceled)

7. The method of claim 1, wherein temperature is at least 100° C.

8. (canceled)

9. The method of claim 7, wherein the medical catheter or tube is exposed to the temperature for 2 hours or less.

10-14. (canceled)

15. The method of claim 4, wherein the drug is a local anesthetic and the local anesthetic includes lidocaine.

16. The method of claim 15, wherein at least at least 2 mg/cm² of lidocaine is embedded in at least a portion of the catheter wall or tube.

17. (canceled)

18. The method of claim 15, wherein a quantity of lidocaine is embedded in at least a portion of the catheter wall or tube such that when the portion is submerged in a 0.15M pH 7.4 phosphate buffer at 37° C., lidocaine is released at rates of at least 1 mcg/hour/cm² continuously for at least 72 hours.

19. The method of claim 1, wherein the drug includes an anti-infection agent.

20. The method in claim 1, wherein the drug includes chlorhexidine.

21-22. (canceled)

23. The method of claim 20, wherein a quantity of the chlorhexidine is embedded in at least a portion of the catheter wall or tube such that when the portion is submerged in a 0.15M pH 7.4 phosphate buffer at 37° C., chlorhexidine is released at rates of at least 0.1 mcg/hour/cm² continuously for at least 72 hours.

24. The method in claim 1, wherein the drug includes both lidocaine and chlorhexidine.

25-28. (canceled)

29. The method in claim 1, wherein more than 50 wt % the drug in the loading solution exists as a unionized species when the catheter is first submerged into the loading solution.

30. (canceled)

31. The method in claim 1, wherein the medical catheter or tube comprises latex.

32-37. (canceled)

38. The method of claim 1, wherein the loading solution comprises at least 90 wt % water and has a pH of at least about 7.

39-60. (canceled)

61. A system for storing a medical catheter or tube with embedded drug, comprising:
a medical catheter or tube embedded with a drug; and
a solution comprising the same drug, wherein at least a portion of the medical catheter or tube is submerged in the solution during storage.

62. (canceled)

63. The system of claim 61, wherein the drug is a local anesthetic and the local anesthetic includes lidocaine.

64. The system of claim 61, wherein the drug includes an anti-infection agent.

65-67. (canceled)

68. The system of claim 61, wherein the medical catheter or tube is a urinary catheter, a medical drainage tube, a feeding tube, a trachea tube, an intravenous catheter, a central venous catheters, an arterial catheter, a peripherally inserted central catheter, an umbilical (arterial or venous) catheter, a gastric tube, a uterine tube, a chest tube, a peritoneal catheter, a renal catheter, a dialysis catheter, or a tissue drainage tube.

69. (canceled)

70. The system of claim 61, wherein the viscosity of the solution is from 300 to 600,000 centipoise

71. A medical catheter or tube capable of releasing drug for at least 24 hours when used in a patient, comprising wall material having a wall area with a wall concentration of drug of at least 1 mg/cm², wherein the drug is diffused into the wall material via a loading solution at a temperature of at least 35° C.

72-81. (canceled)

82. A sterile system of a loading solution and a medical catheter or tube capable of releasing drug for at least 24 hours when used in a patient, comprising:
a medical catheter or tube comprising wall material having a wall surface, and
a loading solution, including a drug, in which at least a portion of the wall surface is submersed,
wherein a first portion of the drug is in the loading solution and a second portion of the drug is absorbed into at least a portion of the wall material at a wall concentration of at least 1 mg/cm², and wherein the medical catheter or tube and the loading solution have been autoclaved together to provide a sterile medical catheter or tube.

83-89. (canceled)

90. The system of claim 82, wherein the drug embedded in the wall material of the medical catheter or tube and in the loading solution are in complete dynamic equilibrium.
